(19) United States
(12) Patent Application Publication
(10) Pub. No.: US 2025/0262132 A1
(43) Pub. Date: Aug. 21, 2025

(54) Pre-Packaged Single-Use on Demand Beauty Care Product Making Method and Apparatus

(71) Applicant: Kenya Eaton, Glen Arm, MD (US)

(72) Inventor: Kenya Eaton, Glen Arm, MD (US)

(21) Appl. No.: 19/057,826

(22) Filed: Feb. 19, 2025

Related U.S. Application Data
(60) Provisional application No. 63/555,215, filed on Feb. 19, 2024.

(51) Int. Cl. A61K 8/02; A61K 8/97; A61Q 5/00

(52) U.S. Cl. CPC A61K 8/0216; A61K 8/97; A61Q 5/00; A61K 2800/652; A61K 2800/805

(57) ABSTRACT

A method for making a pre-packaged single-use on-demand beauty care product comprises providing a beverage filter cartridge containing a dry ingredient mixture, inserting it into a single serve beverage brewer, introducing water, brewing to produce a liquid beauty care product, and dispensing into a collection container. The dry ingredient mixture may include natural ingredients like rice, herbs, and botanicals. The beverage filter cartridge has a higher micron filter than standard paper filters. The brewer may be a Keurig or Nespresso machine, with cartridges compatible with various pod systems. The method allows for convenient production of fresh, preservative-free beauty formulations using existing beverage brewing equipment. Benefits include customization, quality control, and on-demand preparation. The liquid product can be applied to hair and scalp for cleansing, nourishing and moisturizing effects. Overall, the invention provides a novel approach to natural beauty care combining convenience and effectiveness.

### CROSS-REFERENCE TO RELATED APPLICATIONS

This application claims the benefit of U.S. Provisional Application No. 63/555,215, filed Feb. 19, 2024, which is incorporated herein by reference in its entirety.

### FIELD OF THE INVENTION

The present disclosure relates generally to beauty care products and methods of making the same. More particularly, various embodiments are directed to pre-packaged single-use on-demand beauty care product making methods and apparatus.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Post pandemic beauty care industry has seen a rise in DIY consumers. Nielsen IQ reports this transition from salons to homes in support of minimalist beauty ideals held true through the pandemic years and remains a strong factor. Customer interest in self-care is now combined with the idea of self-reliance and holistic well-being versus commercial beauty care products. Thus, beauty care needs convenience, transparency, value, trust, and authenticity play a key role in consumers' beauty care choices.

Commercial beauty care products often utilize harsh chemicals that may strip away natural oils from skin and hair, as well as colors and preservatives that may disrupt the balance of healthy skin microbiome. Artificial colors can potentially lead to irritation, blocked pores, inflammation, sensitize the skin, and in some cases may be associated with more serious conditions. Commercial beauty care products may contain 60-80% water on average which is used to liquify and disperse ingredients. With water readily available to the consumer the addition of water at the manufacturer may become a costly and unnecessary ingredient for the consumer.

DIY beauty care recipes often lack preservatives so if not used immediately the products may lend themselves to the development of bacteria and mold quickly. The preparation areas (kitchen, bathroom) may not be ideal for beauty product creation. Quality ingredients are desirable to help ensure efficacy, consistency, and cleanliness. Fresh store-bought DIY ingredients such as eggs, fruits, and vegetables may contain pathogens in and/or on them that could potentially cause skin problems if not handled properly. Often equipment needed to replicate mass-produced beauty care products are very expensive. Household appliances such as blenders, mixers, and double boilers may be necessary.

It is with respect to these and other considerations that the various embodiments described below are presented.

### BRIEF SUMMARY

The following presents a simplified summary of one or more embodiments of the present disclosure in order to provide a basic understanding of such embodiments. This summary is not an extensive overview of all contemplated embodiments, and is intended to neither identify key or critical elements of all embodiments nor delineate the scope of any or all embodiments. Its sole purpose is to present some concepts of one or more embodiments of the present disclosure in a simplified form as a prelude to the more detailed description that is presented later.

In accordance with one or more embodiments and corresponding disclosure thereof, various aspects are described in connection with a pre-packaged single-use on-demand beauty care product making method and apparatus. In one embodiment, the method may include providing a unique composition which may be combined with various amounts of water. The mixture may be rinsed throughout the hair. The mixture can be used as often as necessary to satisfy the consumer's moisture maintenance routine.

In another embodiment, an apparatus for making a pre-packaged single-use on-demand beauty care product may include a single serve beverage brewer such as a Keurig cup dispenser or a Nespresso Vertuo, and a corresponding beverage filter cartridge/pod design such as Nesspresso Original Line, Nesspresso Virtue, Nescafe Dolce Gusto, Keurig K-cups, Keurig 2.0, K-fee Caffitaly, Lavazza A Medo Mio, Vittoria Esspressotoria, soft pods, or ESE pods.

In yet another embodiment, a system for making a pre-packaged single-use on-demand beauty care product may include a single serve beverage brewer, a beverage filter cartridge/pod containing a dry ingredient mixture, and a collection container for the brewed beauty care product. To the accomplishment of the foregoing and related ends, the one or more embodiments comprise the features hereinafter fully described and particularly pointed out in the claims. The following description and the annexed drawings set forth in detail certain illustrative features of the one or more embodiments. These features are indicative, however, of but a few of the various ways in which the principles of various embodiments may be employed, and this description is intended to include all such embodiments and their equivalents.

### BRIEF DESCRIPTION OF THE DRAWINGS

The disclosed embodiments will hereinafter be described in conjunction with the appended drawings provided to illustrate and not to limit the disclosed embodiments, wherein like designations denote like elements.

FIG. 1 is a diagram illustrating various single serve beverage brewer systems, pod designs, and compatible machines in accordance with an embodiment of the present disclosure.

FIG. 2 is a photograph showing two packages of Zera Rinse beauty care products in accordance with an embodiment of the present disclosure.

FIG. 3 is a method illustrating a setup for making a beauty care product in accordance with an embodiment of the present disclosure.

FIG. 4 is a depiction of a beverage pod in accordance with an embodiment of the present disclosure.

FIG. 5 is a depiction showing various containers holding a brewed beauty care product in accordance with an embodiment of the present disclosure.

FIG. 6 is a method depicting a person applying and using the Zera Rinse beauty care product in a bathtub setting in accordance with an embodiment of the present disclosure.

FIG. 7 is a method depicting a person applying and using the Zera Rinse beauty care product in a bathroom setting in accordance with an embodiment of the present disclosure.

FIG. 8 is a method depicting a person applying and using the Zera Rinse beauty care product in a shower setting in accordance with an embodiment of the present disclosure.

FIG. 9 is a method depicting a person applying and using the Zera Rinse beauty care product in a bathroom setting in accordance with an embodiment of the present disclosure.

FIG. 10 is a flowchart illustrating a process of using a beauty care product in accordance with an embodiment of the present disclosure.

### DETAILED DESCRIPTION

The present disclosure relates to beauty care product making methods and apparatus. More particularly, various embodiments are directed to a unique composition which may be combined with various amounts of water. The mixture may be rinsed throughout the hair. The mixture can be used as often as necessary to satisfy the consumer's moisture maintenance routine. The composition may be produced as a prepackaged single use on demand hair and skin care product with minimal preparation by use of a single serve beverage brewer and beverage filter cartridge/pod.

In the following detailed description, numerous specific details are set forth to provide a thorough understanding of the claimed subject matter. However, it will be understood by those skilled in the art that claimed subject matter may be practiced without these specific details. In other instances, methods, apparatuses or systems that would be known by one of ordinary skill have not been described in detail so as not to obscure claimed subject matter.

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

In one embodiment, the ingredients may be added together and mixed via a single serve beverage brewer such as a Keurig or Nespresso. The formula used may compete with organic “hair tea rinses”. The method of production may utilize an existing household appliance (single serve beverage brewer such as Nespresso Vertuo or Keurig and corresponding beverage filter cartridge/pod) to produce the final product (cleansing rinse and conditioning rinse).

In another embodiment, the dry ingredients may be mixed according to a predetermined formula. The beverage filter cartridge/pod may be filled with the resulting mixture by weight according to the specifications of the beverage filter cartridge/pod. The beverage filter cartridge/pod may be loaded into the corresponding single serve beverage brewer. The beauty care formula may be steeped and/or brewed according to the process of the specific single serve beverage brewer. The resulting liquid may be filtered into a container according to the process of the specific single serve beverage brewer. The resulting liquid (beauty care product) may be allowed to cool before use.

In yet another embodiment, the current disposable beverage filter cartridge/pod and single serve beverage brewer may be used for producing food and beverages only. Additionally, the paper filter used in disposable beverage filter cartridge/pod (i.e., K-cup) may restrict the transfer of beneficial particles in the beauty care preparation. Replacing the paper filter with a higher micron filter may allow for the proper extraction and filtration of the beauty care formula. In still another embodiment, the method of production can be used to produce various beauty care products beyond the present formulas.

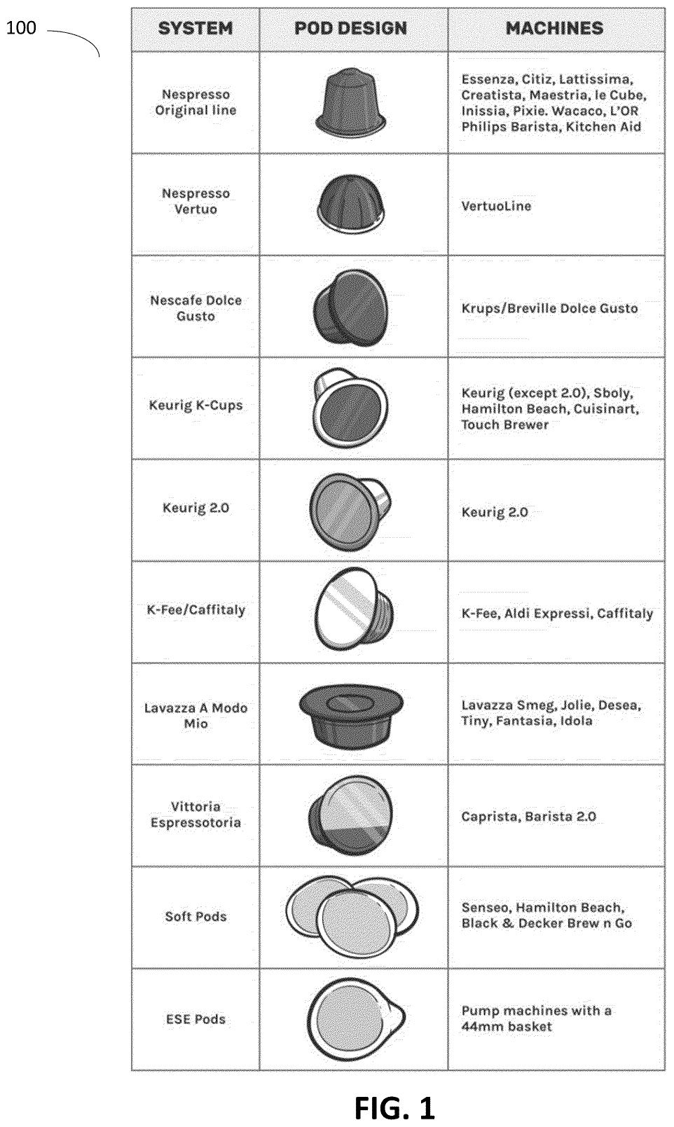

FIG. 1 illustrates various single serve beverage brewer systems, pod designs, and compatible machines in accordance with an embodiment of the present disclosure. The systems may include Nespresso Original line, Nespresso Vertuo, Nescafe Dolce Gusto, Keurig K-Cups, Keurig 2.0, K-Fee/Caffitaly, Lavazza A Modo Mio, Vittoria Espressotoria, Soft Pods, and ESE Pods. Each system may have its own pod design and may be compatible with specific machines.

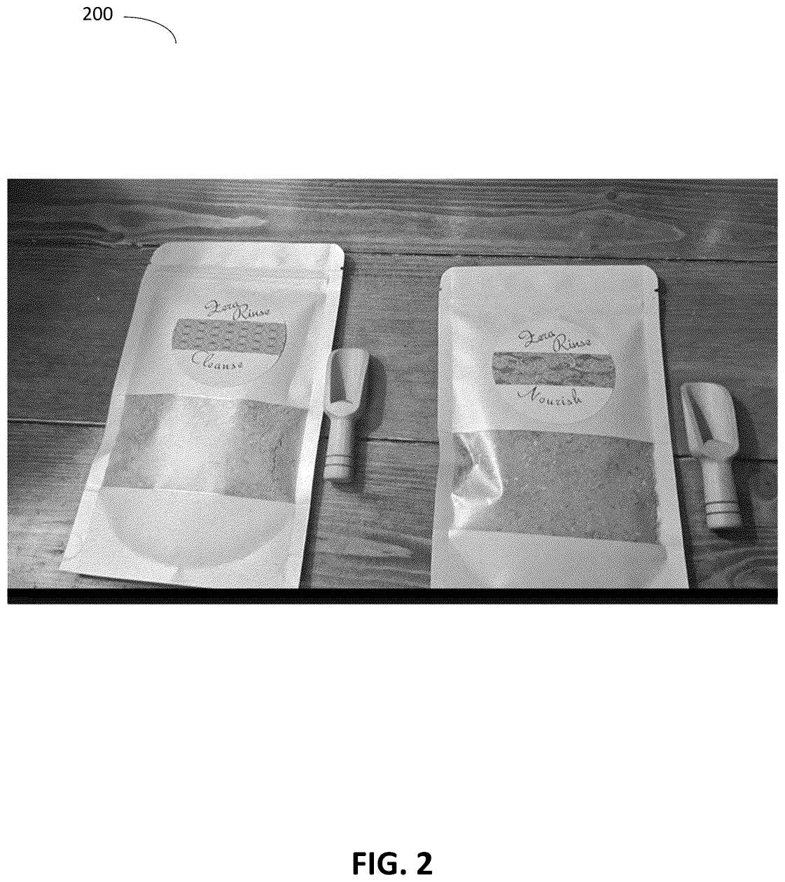

FIG. 2 shows two packages of beauty care products in accordance with an embodiment of the present disclosure. The packages may be labeled “Zera Rinse Cleanse” and “Zera Rinse Nourish”. Each package may contain a brown powder proprietary mixture and may be accompanied by a small wooden scoop.

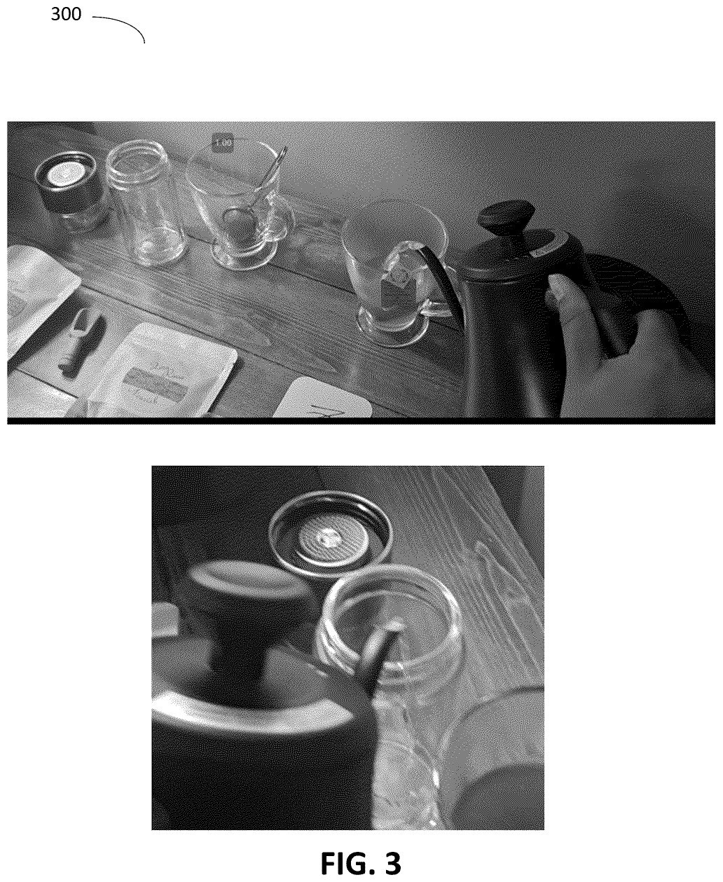

FIG. 3 illustrates a setup for making a beauty care product in accordance with an embodiment of the present disclosure. The setup may include a kettle, glass mugs, and small pouches with scoops and labels related to coffee or tea.

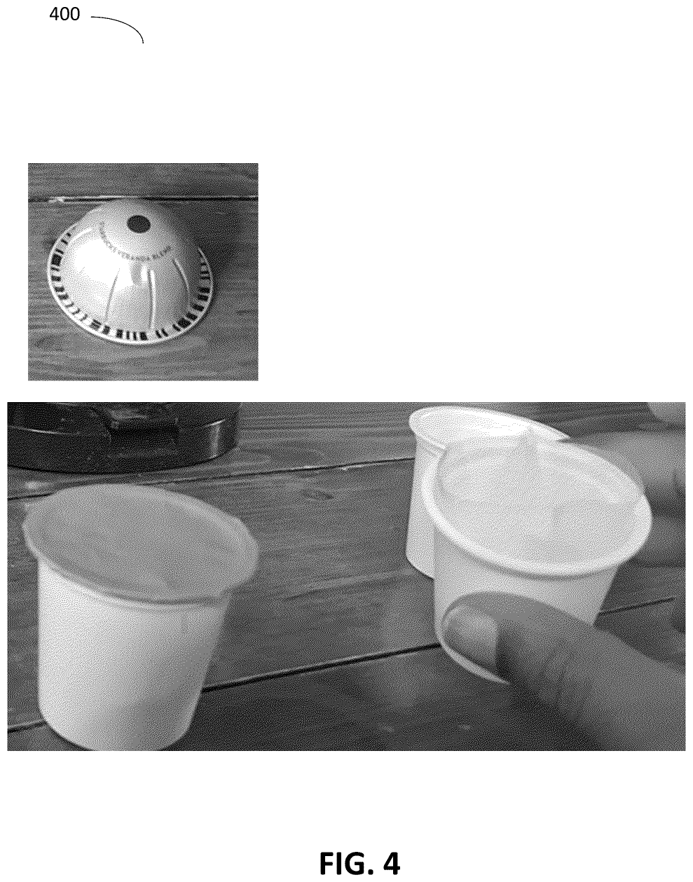

FIG. 4 shows a beverage pod in accordance with an embodiment of the present disclosure. The pod may be dome-shaped with the text “COFFEE & TEA” around its edge.

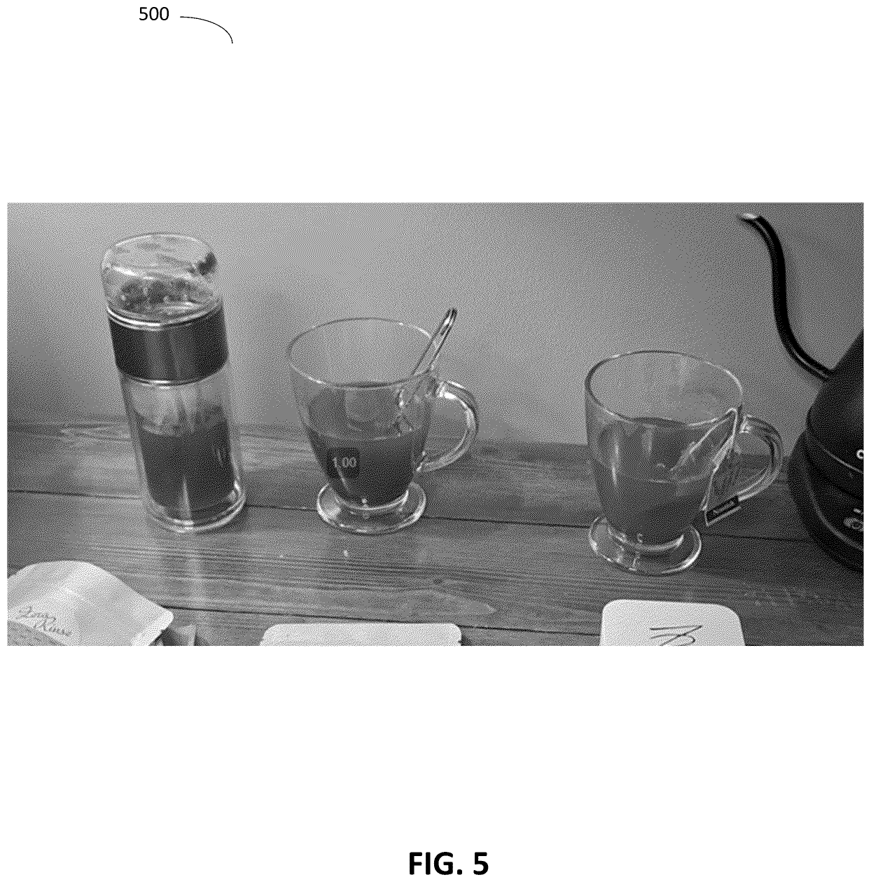

FIG. 5 illustrates various containers holding a brewed beauty care product in accordance with an embodiment of the present disclosure. The containers may include a tall travel mug with a cap, a glass mug with a spoon, and another glass mug with a teabag label visible.

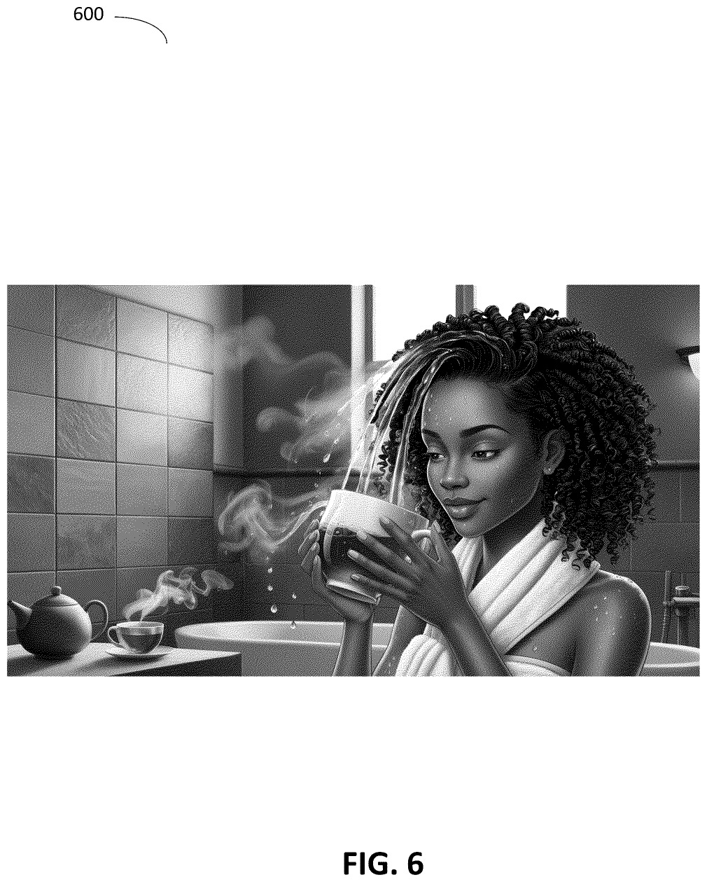

FIG. 6 depicts a person using a beauty care product in a bathtub setting in accordance with an embodiment of the present disclosure. The person is applying the beauty care product to their hair in accordance with in accordance with an embodiment of the present disclosure.

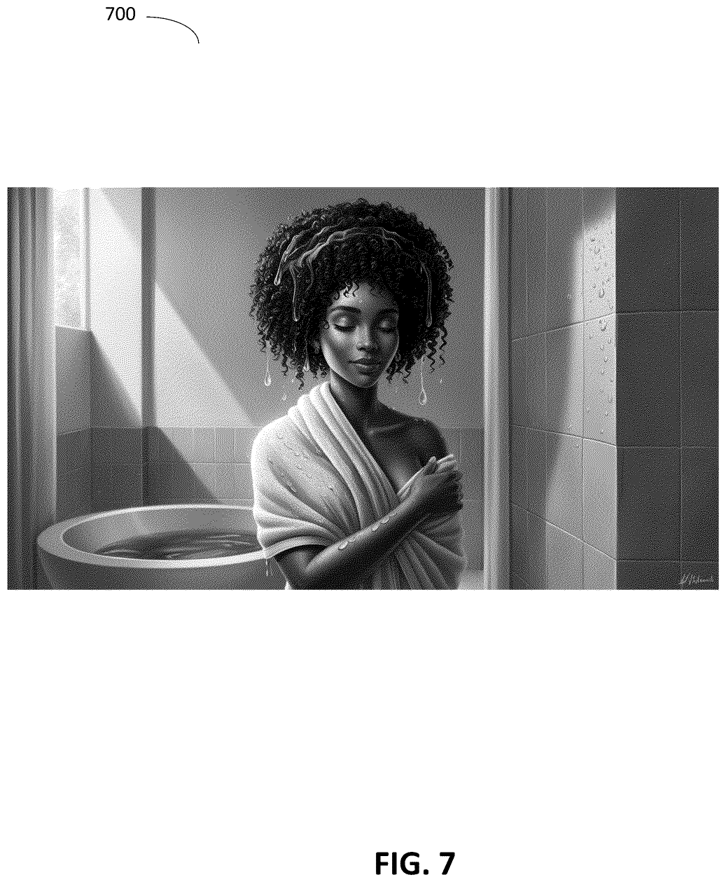

FIG. 7 shows a person after using a beauty care product in a bathroom setting in accordance with an embodiment of the present disclosure. The person is allowing the beauty product to moisturize, cleanse, and nourish the scalp and their hair in accordance with in accordance with an embodiment of the present disclosure.

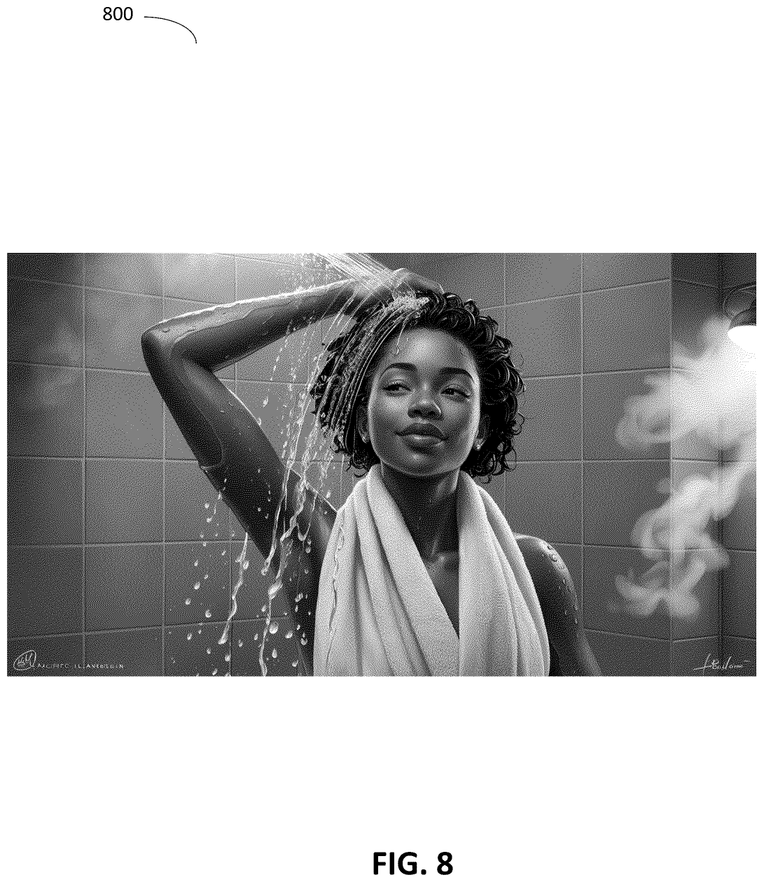

FIG. 8 depicts a person using a beauty care product in a shower setting in accordance with an embodiment of the present disclosure. The person is rinsing the beauty care product out their hair in accordance with in accordance with an embodiment of the present disclosure.

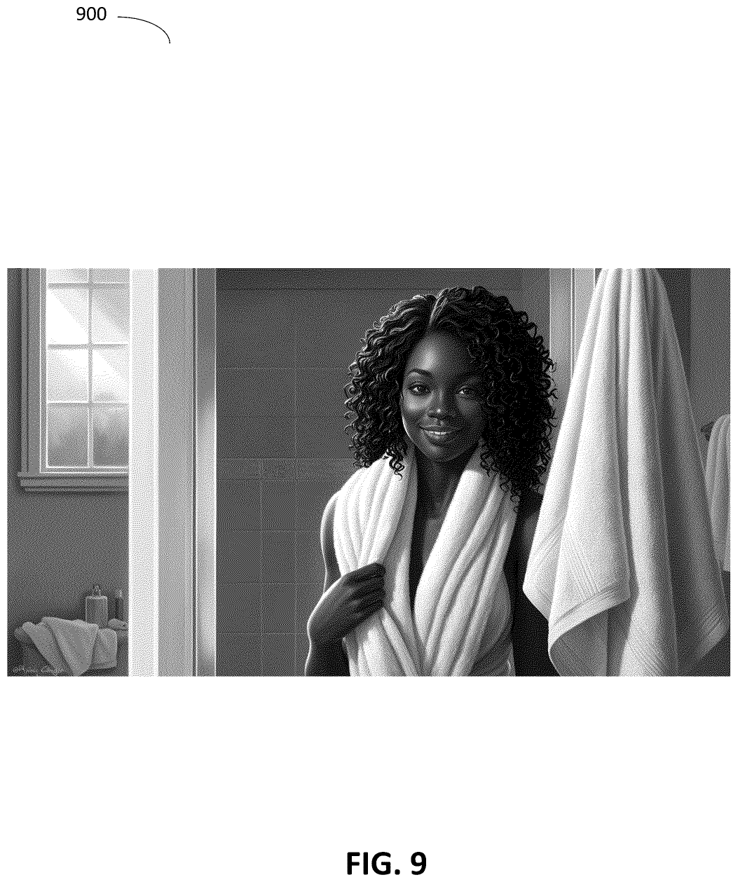

FIG. 9 shows a person after using a beauty care product in a bathroom setting in accordance with an embodiment of the present disclosure. The person is drying their hair and removing the remnants of the beauty care product out of their hair in accordance with in accordance with an embodiment of the present disclosure.

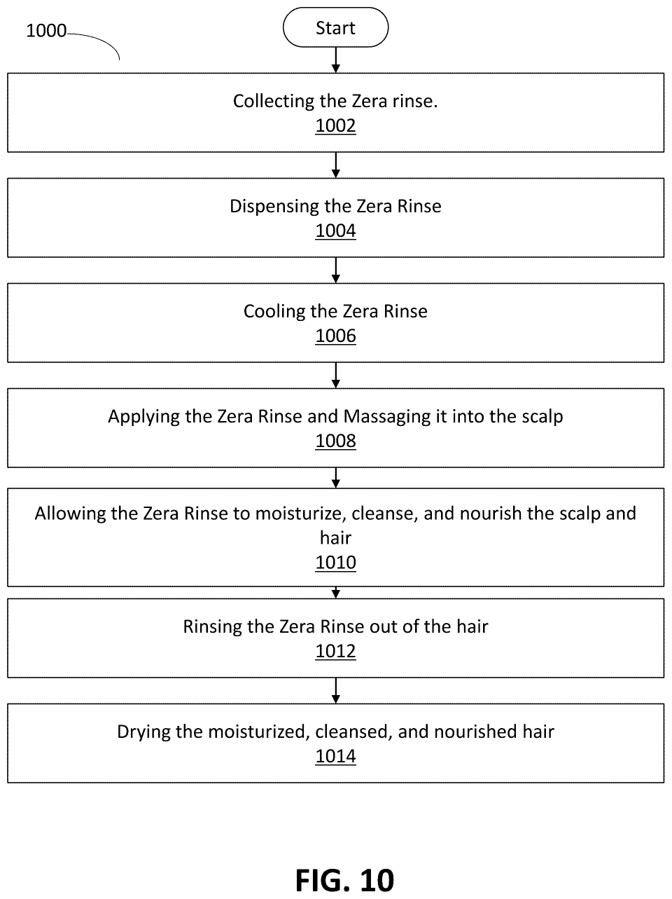

FIG. 10 is a flowchart illustrating a process of using a beauty care product in accordance with an embodiment of the present disclosure. The process may include collecting the Zera rinse (1002), dispensing the Zera Rinse (1004), cooling the Zera Rinse (1006), applying the Zera Rinse and massaging it into the scalp (1008), allowing the Zera Rinse to moisturize, cleanse, and nourish the scalp and hair (1010), rinsing the Zera Rinse out of the hair (1012), and drying the moisturized, cleansed, and nourished hair (1014).

The cleanse and nourish formulas for hair based on natural organic ingredients may provide several benefits. The natural ingredients in these cleanse and nourish formulas can offer multiple advantages for hair care. The gentle cleansing action helps remove impurities without stripping away essential oils, providing a milder alternative to harsh chemical shampoos. The nourishing ingredients like rice, aloe, and various herbs supply nutrients and moisture to strengthen and condition the hair.

Natural components such as shikakai, amla, and bhringraj may promote a healthy scalp environment and potentially stimulate hair growth. The combination of ingredients can help smooth the hair cuticle, potentially reducing frizz and improving overall hair texture. By offering separate cleanse and nourish formulas, users can tailor their hair care routine to their specific needs.

Many of the natural ingredients contain antioxidants that can help protect hair from environmental damage. The pleasant natural scents from ingredients like rose and cinnamon provide an enjoyable sensory experience during use. These formulas appeal to consumers looking to avoid synthetic chemicals in their hair care products.

Some ingredients like aloe and hibiscus may have anti-inflammatory properties that could benefit the scalp. The natural oils and herbs can help improve hair shine and luster. For those with colored hair, these natural ingredients may be gentler and help maintain color vibrancy compared to some chemical-based products.

The combination of ingredients may help balance oil production on the scalp, potentially benefiting both oily and dry hair types. Using natural, organic ingredients appeals to environmentally-conscious consumers. Different natural ingredients may target various hair issues like dryness, brittleness, or lack of volume.

The on-demand brewing method ensures that each application uses freshly prepared product, potentially maximizing the efficacy of the natural ingredients. This approach to hair care provides a customizable, natural alternative that aligns with current trends towards clean beauty and personalized hair treatments.

The cleanse and nourish formulas for hair based on natural organic ingredients may provide several benefits. For instance, the natural ingredients in the formulas may help cleanse and nourish hair without harsh chemicals. Ingredients like rice, hibiscus, rose, and herbs can gently cleanse the hair and scalp while providing nutrients and moisture. This can help maintain the hair's natural oils and pH balance. The organic plant-based ingredients may offer conditioning and moisturizing effects to improve hair texture, shine, and manageability. Components like aloe vera, flax seeds, and hemp seeds can coat the hair shaft to lock in moisture and add smoothness.

Antioxidants and vitamins present in ingredients like amla, bhringraj, and hibiscus may help nourish the hair follicles and promote healthy hair growth. These natural compounds can strengthen hair from the roots. The herbal ingredients may have antimicrobial and anti-inflammatory properties that can soothe the scalp, reduce dandruff, and address other scalp issues. Shikakai, neem, and tea tree oil are examples of ingredients with these benefits. Using natural ingredients avoids potentially harsh sulfates, silicones, and artificial fragrances that can strip and damage hair over time. This allows for gentler, more frequent use of the hair care products.

In accordance with an embodiment of the present disclosure, the method of brewing and application to hair and scalp offers tremendous benefits over many prior the brewing process of steeping the dry ingredients in hot water may help extract beneficial compounds and essential oils from the herbs and botanicals. This can enhance the potency and effectiveness of the active ingredients. The hot brewing process may help open up the hair cuticles temporarily, allowing for better penetration and absorption of the nourishing ingredients into the hair shaft. The warm temperature of the freshly brewed rinse may improve blood circulation in the scalp when applied, potentially stimulating hair follicles.

Using freshly brewed formulas eliminates the need for preservatives and allows for on-demand preparation of active ingredients. This maintains potency and freshness of the natural compounds. The brewing method creates a liquid formula that can be easily and evenly distributed through the hair for thorough cleansing and nourishment from roots to ends. Beyond hair benefits, the natural herbal ingredients may provide aromatherapy effects to promote relaxation and wellbeing during the hair care routine. The customizable brewing process allows users to adjust the strength and concentration of the formula to suit their individual hair needs and preferences. Overall, this natural brewing method for hair care formulas may provide a holistic approach to maintaining healthy, nourished hair using the power of organic botanicals and herbs. The process aims to cleanse, condition, and treat hair gently yet effectively.

The dry ingredient mixture may comprise various combinations of natural ingredients to create novel cleanse and nourish products for hair and scalp care. For the cleanse formula, ingredients such as rice powder, shikakai powder, amla powder, hibiscus powder, rose petal powder, neem leaf powder, and fenugreek seed powder may be combined in varying ratios. This formula gently cleanses the scalp and hair, removes buildup, balances pH, strengthens hair, promotes growth, and adds shine.

The nourish formula may include ingredients like bhringraj powder, brahmi powder, aloe vera powder, fenugreek seed powder, amla powder, hibiscus powder, and marshmallow root powder in different proportions. This formula deeply conditions, moisturizes, reduces frizz, promotes growth, strengthens hair, and improves scalp health.

Additional ingredients that may be incorporated in various mixtures include ashwagandha powder, gotu kola powder, beet root powder, spirulina powder, alfalfa powder, kelp powder, flaxseed powder, oat straw powder, horsetail powder, slippery elm bark powder, calendula flower powder, chamomile flower powder, lavender flower powder, rosemary leaf powder, sage leaf powder, and nettle leaf powder. These may be added in smaller amounts to target specific hair and scalp concerns.

The dry ingredient mixtures can be formulated to address a range of hair and scalp issues such as cleansing, clarifying, deep conditioning, moisturizing, promoting growth, strengthening, repairing damage, balancing oily scalps, soothing dry/itchy scalps, adding shine, reducing frizz, enhancing curls, and protecting color-treated hair. The single-serve beverage brewing system allows for customization and on-demand preparation of a variety of novel cleanse and nourish products tailored for different hair types, textures, and concerns.

The dry ingredient mixture may comprise various combinations of natural ingredients to create novel cleanse and nourish products for hair and scalp care. Some potential ingredient combinations for a Cleanse Formula may include 30-50% rice powder, 15-25% yerba mate, 5-10% shikakai powder, 5-10% amla powder, 5-10% aritha powder, 2-5% hibiscus powder, 2-5% rose petal powder, and 1-3% aamchur powder. A Nourish Formula may contain 20-30% rice powder, 20-30% flaxseed powder, 20-30% hemp seed powder, 10-15% yerba mate, 5-10% bhringraj powder, 5-10% aloe vera powder, 2-5% hibiscus powder, 2-5% rose petal powder, and 1-3% cinnamon powder. These natural ingredient blends are designed to provide cleansing, nourishing, and conditioning benefits for the hair and scalp when brewed into a liquid product using a single-serve beverage brewing system.

The dry ingredient mixtures may contain anywhere from 3-15 different ingredients in various proportions. Some additional ingredients that may be incorporated include fenugreek seed powder (1-5%), neem leaf powder (1-3%), brahmi powder (2-5%), moringa leaf powder (2-5%), ashwagandha powder (1-3%), licorice root powder (1-3%), marshmallow root powder (1-3%), slippery elm bark powder (1-3%), bentonite clay (5-10%), and kaolin clay (5-10%). These ingredients can be added to the base formulations to provide additional benefits for hair and scalp health. Fenugreek seed powder is rich in proteins and nicotinic acid that can help strengthen hair follicles and promote hair growth. Neem leaf powder has antibacterial and antifungal properties that may help combat scalp infections and dandruff. Brahmi powder is known for its ability to strengthen hair roots and promote hair growth. Moringa leaf powder is high in vitamins and minerals that nourish the hair and scalp. Ashwagandha powder may help reduce hair loss by reducing cortisol levels in the body. Licorice root powder has anti-inflammatory properties that can soothe an irritated scalp. Marshmallow root powder forms a protective coating on the hair shaft to lock in moisture. Slippery elm bark powder adds slip and detangling properties to the formula. Bentonite clay and kaolin clay can help absorb excess oils from the scalp and hair while gently cleansing. The clays also provide minerals that may strengthen the hair.

The dry ingredients may comprise at least one of rice, yerba, hibiscus, rose, shikakai, amla, aritha, aamchur, flax, hemp, bhringraj, aloe, or cinnamon. In some embodiments, the dry ingredients may include 10-40% by weight rice, 5-25% by weight yerba mate, 1-5% by weight hibiscus, 1-5% by weight rose petals, 5-15% by weight shikakai, 5-15% by weight amla, 5-15% by weight aritha, and 1-5% by weight aamchur.

The cleansing formulation may comprise approximately 49% rice, 22% yerba mate, 2% hibiscus, 2% rose petals, 8% shikakai, 8% amla, 8% aritha, and 2% aamchur. The nourishing formulation may comprise approximately 21% rice, 10% yerba mate, 2% hibiscus, 2% rose petals, 21% flax seeds, 21% hemp seeds, 7% bhringraj, 14% aloe, and 2% cinnamon.

Other potential ingredients that may be included in various combinations and amounts include marshmallow root, slippery elm bark, fenugreek seeds, chamomile flowers, lavender buds, rosemary leaves, nettle leaf, horsetail, sage leaf, peppermint leaf, calendula flowers, burdock root, ginger root, turmeric root, licorice root, ashwagandha root, brahmi leaf, neem leaf, tulsi leaf, moringa leaf, green tea leaf, bamboo extract, silica, biotin, panthenol, hydrolyzed proteins, amino acids, plant oils, essential oils, clays, salts, and vitamins.

The beverage filter cartridge may comprise a higher micron filter than a standard paper filter. For example, the filter may have a micron rating between 50-200 microns to allow beneficial smaller particles to pass through while retaining larger plant pieces. The cartridge may be made of materials such as polylactic acid (PLA), nylon, or other food-safe plastics compatible with hot water brewing.

The method may further comprise allowing the liquid beauty care product to cool before use. In some embodiments, the liquid product may be applied to hair and skin while still warm to enhance absorption. The single serve beverage brewer may be a Keurig, Nespresso, or other compatible machine.

Various pod designs may be used, including those compatible with Nespresso Original Line, Nespresso Vertuo, Nescafe Dolce Gusto, Keurig K-cups, Keurig 2.0, K-fee Caffitaly, Lavazza A Modo Mio, Vittoria Esspressotoria, soft pods, or ESE pods. This allows the product to be used with a wide range of existing home brewing systems.

The method provides several advantages over traditional beauty products and DIY recipes. It eliminates the need for preservatives and harsh chemicals, provides precise portioning of ingredients, ensures quality and consistency, reduces preparation time and equipment needs, and allows for customization of formulas. The single-serve brewing process also helps maintain product freshness and potency.

By incorporating these additional ingredients in various combinations and proportions, the dry ingredient mixtures can be customized to target specific hair and scalp concerns. The ingredients may be combined in different ratios to target specific hair and scalp concerns. For example, a mixture for oily hair and scalp may contain higher percentages of shikakai, aritha, and bentonite clay. A mixture for dry, damaged hair may have higher amounts of aloe vera, flaxseed, and marshmallow root.

The dry ingredient mixtures provide various benefits for hair and scalp health. These mixtures offer gentle cleansing without stripping away natural oils, helping to maintain the scalp's natural moisture balance. They work to balance the pH of the scalp, creating an optimal environment for healthy hair growth. The ingredients nourish and moisturize both the hair and scalp, providing essential nutrients and hydration. These formulations are designed to promote hair growth and increase thickness, addressing common concerns like thinning hair or slow growth. They can help reduce dandruff and soothe scalp irritation, alleviating itchiness and flakiness. The mixtures add shine and manageability to the hair, improving its overall appearance and texture.

By strengthening the hair shaft, these ingredients help prevent breakage and split ends, leading to healthier, longer hair over time. The anti-inflammatory properties of certain components work to soothe any inflammation on the scalp, creating a healthier environment for hair follicles. Additionally, the antioxidants present in the mixtures provide protection against environmental damage and oxidative stress, which can negatively impact hair health. Overall, these carefully formulated dry ingredient mixtures offer a comprehensive approach to hair and scalp care, addressing multiple concerns simultaneously while promoting overall hair health and vitality.

The exact formulations may be adjusted based on factors like hair type, scalp condition, desired benefits, and sensory preferences. The dry powders may be packaged in single-use quantities of 10-30 grams per beverage filter cartridge. When brewed with hot water in a single serve beverage machine, the ingredients may create a liquid hair rinse or treatment that can be applied to hair and scalp.

In one or more embodiments, the invention may provide several technical advantages over existing solutions for producing beauty care products. Some potential advantages include: Improved convenience: The use of a single serve beverage brewer and specialized cartridges may allow users to easily produce fresh beauty care products on demand with minimal preparation or cleanup required. This may be more convenient than traditional DIY methods or purchasing pre-made products. Enhanced freshness and quality: By producing small batches on demand, the invention may allow users to create fresher products without the need for preservatives or long shelf life. This may result in higher quality and more effective beauty treatments compared to mass-produced commercial products. Customization capabilities: The system may allow for easy customization of formulations by using different cartridges or adjusting brewing parameters. This could enable users to tailor products to their specific needs. Improved safety and hygiene: Using a closed brewing system and pre-packaged ingredients may reduce contamination risks compared to traditional DIY beauty recipes made in kitchens or bathrooms. The controlled brewing process may also help ensure proper extraction and mixing of ingredients. Resource efficiency: By eliminating water as a pre-added ingredient and allowing users to add it during brewing, the invention may reduce shipping weights and packaging waste compared to traditional liquid beauty products. Consistent results: The controlled brewing process and pre-measured ingredients may allow for more consistent product quality compared to manual DIY methods, while still providing a customized experience. Multi-purpose functionality: The ability to use existing single serve beverage brewers for both beverages and beauty products may increase the utility and value of these common household appliances. Reduced equipment needs: By leveraging existing beverage brewing systems, the invention may eliminate the need for separate specialized equipment for producing beauty products at home.

In one or more instances, some alternative embodiments or variations of the invention may include: Different formulations: In addition to the disclosed cleansing and nourishing hair rinses, the system could be adapted for other beauty products like face masks, body scrubs, or aromatherapy treatments. Adjustable brewing parameters: The system could allow users to customize water temperature, brewing time, or water volume to further tailor the resulting products. Multi-chamber cartridges: Cartridges with separate chambers for different ingredients could allow for more complex formulations or the ability to adjust ratios. Subscription service:

A subscription model could provide users with regular shipments of cartridges tailored to their beauty routines. Smart brewing system: Integration with mobile apps or smart home systems could allow for automated brewing schedules, usage tracking, or personalized recommendations. Eco-friendly packaging: Development of biodegradable or reusable cartridges could further reduce the environmental impact of the system. Professional salon versions: Larger capacity systems could be developed for use in salons or spas to produce fresh treatments for multiple clients. Combination products: Cartridges that produce both a beauty treatment and a complementary beverage (e.g. a hair mask and herbal tea) could provide a more holistic self-care experience.

While the foregoing is directed to embodiments of the present invention, other and further embodiments of the invention may be devised without departing from the basic scope thereof, and the scope thereof is determined by the claims that follow.

The pre-packaged single-use on-demand beauty care product making method and apparatus may provide several technical advantages over existing products and methods. By utilizing pre-measured dry ingredients in sealed cartridges, the invention may ensure consistent quality and freshness of ingredients compared to DIY methods using store-bought items. This may result in more reliable and effective beauty treatments. The sealed cartridges and automated brewing process may reduce contamination risks associated with manual preparation of beauty products in home environments. This may lead to safer products with lower risk of bacterial growth or ingredient spoilage.

The dry ingredient formulations stored in sealed cartridges may have significantly longer shelf life compared to pre-mixed liquid beauty products, eliminating the need for preservatives and extending usability. The single-serve brewer system may provide more accurate and consistent dosing of ingredients and water compared to manual DIY methods. This may result in more reliable product performance across multiple uses. By leveraging existing single-serve brewing technology, the invention may greatly simplify the preparation of beauty products compared to traditional DIY methods requiring multiple tools and steps. This may save time and reduce cleanup.

The cartridge-based system may allow for easy customization of formulations to address different hair and skin needs without requiring separate full-size products. Single-use cartridges may result in less product waste compared to larger bottles of pre-mixed commercial products that often expire before being fully used. The specialized higher micron filter in the cartridges may allow for more efficient extraction of beneficial compounds from the dry ingredients compared to standard paper filters used in beverage applications. By brewing beauty products on-demand, the invention may provide fresher, more potent formulations compared to pre-mixed commercial products that may degrade over time on store shelves. The invention leverages widely available single-serve brewing machines for a new application, potentially increasing the utility and value of equipment many consumers already own.

In conclusion, the pre-packaged single-use on-demand beauty care product making method and apparatus described herein may provide several advantages over conventional beauty care products and DIY methods. The system may allow for convenient production of fresh, preservative-free beauty care formulations using existing single-serve beverage brewing equipment. The specialized beverage filter cartridges containing dry ingredient mixtures may enable consistent, high-quality results while eliminating the need for measuring and mixing ingredients. By utilizing natural ingredients and avoiding harsh chemicals, the system may appeal to consumers seeking more holistic beauty care options. The on-demand nature of production may help ensure product freshness and reduce waste compared to pre-made commercial products with limited shelf life. Overall, this invention may offer a novel approach to beauty care that combines the benefits of natural ingredients, convenience, customization, and quality control in an accessible format for home use.

What is claimed is:

1. A method for making a pre-packaged single-use on-demand beauty care product, the method comprising:
providing a beverage filter cartridge containing a dry ingredient mixture;
inserting the beverage filter cartridge into a single serve beverage brewer;
introducing water into the single serve beverage brewer;
brewing the dry ingredient mixture to produce a liquid beauty care product; and
dispensing the liquid beauty care product into a collection container.

2. The method of claim 1, wherein the dry ingredient mixture comprises at least one of rice, yerba, hibiscus, rose, shikakai, amla, aritha, aamchur, flax, hemp, bhringraj, aloe, or cinnamon.

3. The method of claim 1, wherein the beverage filter cartridge comprises a higher micron filter than a standard paper filter.

4. The method of claim 1, further comprising allowing the liquid beauty care product to cool before use.

5. The method of claim 1, wherein the single serve beverage brewer is one of a Keurig or Nespresso machine.

6. The method of claim 1, wherein the beverage filter cartridge is compatible with at least one of Nespresso Original Line, Nespresso Vertuo, Nescafe Dolce Gusto, Keurig K-cups, Keurig 2.0, K-fee Caffitaly, Lavazza A Modo Mio, Vittoria Esspressotoria, soft pods, or ESE pods.

7. A system for making a pre-packaged single-use on-demand beauty care product, the system comprising:
a single serve beverage brewer;
a beverage filter cartridge containing a dry ingredient mixture, the beverage filter cartridge configured to be inserted into the single serve beverage brewer; and
a collection container for receiving a liquid beauty care product produced by the single serve beverage brewer.

8. The system of claim 7, wherein the dry ingredient mixture comprises at least one of rice, yerba, hibiscus, rose, shikakai, amla, aritha, aamchur, flax, hemp, bhringraj, aloe, or cinnamon.

9. The system of claim 7, wherein the beverage filter cartridge comprises a higher micron filter than a standard paper filter.

10. The system of claim 7, wherein the single serve beverage brewer is one of a Keurig or Nespresso machine.

11. The system of claim 7, wherein the beverage filter cartridge is compatible with at least one of Nespresso Original Line, Nespresso Vertuo, Nescafe Dolce Gusto, Keurig K-cups, Keurig 2.0, K-fee Caffitaly, Lavazza A Modo Mio, Vittoria Esspressotoria, soft pods, or ESE pods.

12. A method for making a pre-packaged single-use on-demand beauty care product, the method comprising:
providing a beverage filter cartridge containing a dry ingredient mixture;
inserting the beverage filter cartridge into a single serve beverage brewer;
introducing water into the single serve beverage brewer;
brewing the dry ingredient mixture to produce a liquid beauty care product;
dispensing the liquid beauty care product into a collection container;
allowing the liquid beauty care product to cool;
applying the cooled liquid beauty care product to hair;
massaging the cooled liquid beauty care product into a scalp;
allowing the cooled liquid beauty care product to moisturize, cleanse, and nourish the scalp and hair;
rinsing the cooled liquid beauty care product out of the hair; and
drying the moisturized, cleansed, and nourished hair.

13. The method of claim 12, wherein the dry ingredient mixture comprises at least one of rice, yerba, hibiscus, rose, shikakai, amla, aritha, aamchur, flax, hemp, bhringraj, aloe, or cinnamon.

14. The method of claim 12, wherein the beverage filter cartridge comprises a higher micron filter than a standard paper filter.

15. The method of claim 12, wherein the single serve beverage brewer is one of a Keurig or Nespresso machine.

16. The method of claim 12, wherein the beverage filter cartridge is compatible with at least one of Nespresso Original Line, Nespresso Vertuo, Nescafe Dolce Gusto, Keurig K-cups, Keurig 2.0, K-fee Caffitaly, Lavazza A Modo Mio, Vittoria Esspressotoria, soft pods, or ESE pods.

17. The method of claim 12, wherein the beverage filter cartridge comprises:
an outer casing compatible with the single serve beverage brewer;
a filter within the outer casing, wherein the filter has a higher micron rating than a standard paper filter; and
the dry ingredient mixture contained within the filter.

18. The method of claim 12, further comprising providing a kit comprising:
a plurality of the beverage filter cartridges, each containing the dry ingredient mixture;
instructions for using the beverage filter cartridges with the single serve beverage brewer to produce the liquid beauty care product; and
the collection container for receiving the liquid beauty care product.

19. The method of claim 12, wherein the dry ingredient mixture is formulated to provide at least one of cleansing, conditioning, moisturizing, nourishing, strengthening, or repairing effects to the hair and scalp.

20. The method of claim 12, wherein brewing the dry ingredient mixture comprises extracting beneficial compounds and essential oils from herbs and botanicals in the dry ingredient mixture to enhance potency and effectiveness of active ingredients in the liquid beauty care product.
